(19) United States
(12) Patent Application Publication
(10) Pub. No.: US 2008/0138816 A1
(43) Pub. Date: Jun. 12, 2008

(54) METHODS FOR PREPARING AND ANALYZING CELLS HAVING CHROMOSOMAL ABNORMALITIES

(75) Inventors: James Brawley, Seattle, WA (US); Philip J. Morrissey, Seattle, WA (US); Rosalynde J. Finch, Seattle, WA (US); David A. Basiji, Seattle, WA (US); Luchuan Liang, Woodinville, WA (US)

Correspondence Address: SEED INTELLECTUAL PROPERTY LAW GROUP PLLC, 701 FIFTH AVE, SUITE 5400, SEATTLE, WA, 98104, US

(73) Assignee: AMNIS CORPORATION, Seattle, WA (US)

(21) Appl. No.: 11/838,138

(22) Filed: Aug. 13, 2007

Related U.S. Application Data
(62) Division of application No. 11/134,243, filed on May 20, 2005.

(51) Int. Cl. C12Q 1/68; C12Q 1/04

(52) U.S. Cl. 435/6; 435/34

(57) ABSTRACT

The present invention provides methods for preparing cells with highly condensed chromosomes, such as sperm, and methods for detecting and quantifying specific cellular target molecules in intact cells. Specifically, methods are provided for detecting chromosomes and chromosomal abnormalities, including aneuploidy, in intact cells using fluorescence in situ hybridization of cells in suspension, such as sperm cells.

### CROSS-REFERENCES TO RELATED APPLICATIONS

This application is a divisional of U.S. patent application Ser. No. 11/134,243, filed May 20, 2005, now pending, which claims the benefit of U.S. Provisional Patent Application No. 60/573,775 filed May 20, 2004. These applications are herein incorporated by reference in their entireties.

### STATEMENT OF GOVERNMENT INTEREST

This invention was made with government support under Contract No. N43-ES-35507 awarded by the National Institute for Environmental Health Sciences (NIEHS). The government may have certain rights in this invention.

### TECHNICAL FIELD

The present invention relates generally to methods for detecting and quantifying specific cellular target molecules in intact cells by multispectral imaging of the cells in flow, and more specifically, to methods of processing an intact cell to facilitate fluorescence in situ hybridization of cells, such as sperm cells, in suspension.

### BACKGROUND

Aneuploidy, the condition of having more than or less than the normal number of chromosomes, is the most common class of cytogenetic abnormality in humans, occurring in at least 0.3% of live births, approximately 4% of stillbirths, and 35% of spontaneous abortions (Griffin, Int. Rev. Cytology 167:263, 1996). The few aneuploidies that survive to birth include trisomy 21 (Down syndrome); trisomy 13 (Patau's syndrome); trisomy 18 (Edward's syndrome); trisomy 8 (Warkany Syndrome 2); XXX; 45,X (Turner syndrome); XXY (Klinefelter syndrome); and XYY. The majority of autosomal aneuploidies at birth have been determined to be maternal in origin (Hassold and Sherman, The origin of nondysjunction in humans, in: A. T. Sumner and A. C. Chandley (Eds.) Chromosome's Today, Chapman Hall, New York, N.Y., Vol 11, pp. 313-322, 1993), whereas the majority of sex-chromosomal aneuploidies at birth involve paternal chromosomes (100% for XYY, 80% for X0, 50% for XXY, and 10% for XXX) (Chandley, J. Med. Genetics 28:217, 1991).

Despite the frequency of aneuploidy and its burden to human health, very little is known regarding the genetic, physiological, and environmental risk factors for that may induce germ line aneuploidy. Recent work using fluorescence in-situ hybridization (FISH) with chromosome specific probes has allowed researchers to quantify the frequency and type of aneuploidy of male germ cells (Martin et al., Cytogen. Cell Genetics 64:23, 1993; Robbins et al., Am J. Human Genetics 52:799, 1993; Robbins et al., Reprod. Fertil. Dev. 7:799, 1995; Wyrobek et al., Am. J. Human Genetics 53:1, 1994). This sperm-FISH assay uses chromosome-specific DNA probes labeled with different fluorochromes. These probes are hybridized to sperm DNA and the fluorescent signals are evaluated for each cell using a fluorescence microscope. A positive signal represents the presence of that particular chromosome.

Sperm-FISH analysis has been applied towards measuring aneuploidy rates of human sperm (Robbins et al., Am. J. Human Genetics 55:A68; 1994; Wyrobek et al., Am. J. Human Genetics 57:A131, 1995; Robbins et al., Environ. Mol. Mutagen. 30:175, 1997; Robbins et al., Reprod. Fertil. Dev. 7:799, 1995; Levron et al., Fertil Steril 76:479, 2001). Given the difficulties in conducting human exposure studies, efforts have been made to develop experimental animal models to identify substances that increase sperm cell aneuploidy, and to better understand the induction and persistence of sperm aneuploidy. The most developed of these is the mouse (murine) model, which is similar to the three-chromosome human sperm-FISH assay (Lowe et al., Chromosoma 105:204, 1996).

Despite the development of animal models, the study of germ line aneuploidy remains a slow, labor-intensive, and data-sparse process due to the limitations of the manual scoring of sperm-FISH assays (Schmid et al., Mutagenesis 16:189, 2001). A typical assay requires the scoring of approximately 10,000 cells, which takes approximately one week (given the manual scoring rates of approximately 1,000 per hour, see Baumgartner et al. (Cytometry 44:156, 2001), and the necessity for two independent scores, see Schmid et al., 2001). To date, the only automated scoring technique reported has been one using a laser-scanning cytometer (LSC) to assess aneuploidy of sperm-FISH on microscope slides (see, e.g., Baumgartner et al., 2001). This technique, as described, is limited for the following reasons: (i) the assessment of aneuploidy is based upon intensity integration of the fluorescent probes rather than the more precise discrete FISH spot detection and enumeration employed in manual scoring; and (ii) the LSC technique uses an air coupled optical objective that limits both its field of view and image quality.

Thus, a need is recognized in the art for techniques that permit detection and quantitation of aneupolidy in cells in flow, which would provide an opportunity to study suspension-based cell lines and primary cells. Furthermore, methods for preparing cells in suspension for multispectral analysis, such as sperm cells, are needed. The present invention meets such needs and further provides other related advantages.

### BRIEF SUMMARY OF THE INVENTION

The present invention is directed to methods for determining the presence of chromosomes and/or the presence of chromosomal abnormalities in cells (somatic and germ cells) using multispectral imaging of the cells in flow. In a particular embodiment, the methods relate to detecting chromosomes and chromosomal abnormalities in intact sperm cells using multispectral imaging of the cells in flow.

In one embodiment, a method is provided for detecting a chromosome in a sperm cell, comprising (a) contacting the sperm cell with a nucleic acid probe that is capable of hybridizing to a target chromosomal DNA sequence, under conditions and for a time sufficient to permit interaction of the chromosomal DNA in the sperm cell and the probe; and (b) detecting the probe hybridized to the chromosomal DNA by multispectral imaging of the sperm cell in flow. In a certain embodiment, the sperm cell is a human sperm cell, and in another embodiment, the sperm cell is a sperm cell from a non-human animal. In a particular embodiment, the probe is detectably labeled with a reporter molecule. In certain embodiments, the reporter molecule is a fluorochrome, and in other certain embodiments, the reporter molecule is biotin. In certain embodiments of the method, wherein the reporter molecule is biotin, the method further comprises contacting the cell with streptavidin conjugated to a fluorochrome.

In a specific embodiment of this method for detecting a chromosome in a sperm cell, the sperm cell is contacted with (a) a first nucleic acid probe and a second nucleic acid probe; (b) a first nucleic acid probe, a second nucleic acid probe, and a third nucleic acid probe; or (c) a first nucleic acid probe, a second nucleic acid probe, a third nucleic acid probe, and a fourth nucleic acid probe, wherein the first nucleic acid probe is capable of hybridizing to a first target chromosomal DNA sequence, wherein the second nucleic acid probe is capable of hybridizing to a second target chromosomal DNA sequence, wherein the third nucleic acid probe is capable of hybridizing to a third target chromosomal DNA sequence, and wherein the fourth nucleic acid probe is capable of hybridizing to a fourth target chromosomal DNA sequence. In a further embodiment, the first probe is detectably labeled with a first reporter molecule, the second probe is detectably labeled with a second reporter molecule, the third probe is detectably labeled with a third reporter molecule, and the fourth probe is detectably labeled with a fourth reporter molecule. In one embodiment, the first reporter molecule is a first fluorochrome, the second reporter molecule is a second fluorochrome, the third reporter molecule is a third fluorochrome, and the fourth reporter molecule is a fourth fluorochrome. In another embodiment, one or more of the first reporter molecule, the second reporter molecule, the third reporter molecule, and the fourth reporter molecule is biotin. In certain embodiments of the method, wherein a reporter molecule is biotin, the method further comprises contacting the cell with streptavidin conjugated to a fluorochrome.

In another embodiment, a method is provided for determining the presence of a chromosomal abnormality in a cell, comprising (a) contacting the cell with a nucleic acid probe that is capable of hybridizing to a target chromosomal DNA sequence, under conditions and for a time sufficient to permit interaction of the chromosomal DNA in the cell and the probe; (b) detecting the hybridized probe by multispectral imaging of the cell in flow; and (c) comparing the multispectral imaging of the cell in flow to the multispectral imaging of a chromosomally normal cell in flow, and thereby determining the presence of a chromosomal abnormality in the cell. In a particular embodiment, the chromosomal abnormality detected is aneuploidy, chromosomal translocation, chromosomal inversion, gene amplification, gene mutation, or gene deletion. In a particular embodiment, the probe is detectably labeled with a reporter molecule. In certain embodiments, the reporter molecule is a fluorochrome, and in other certain embodiments, the reporter molecule is biotin. In certain embodiments, when the reporter molecule is biotin, the method further comprises contacting the cell with streptavidin conjugated to a fluorochrome.

In a specific embodiment of this method for determining the presence of a chromosomal abnormality in a cell, the cell is contacted with (a) a first nucleic acid probe and a second nucleic acid probe; (b) a first nucleic acid probe, a second nucleic acid probe, and a third nucleic acid probe; or (c) a first nucleic acid probe, a second nucleic acid probe, a third nucleic acid probe, and a fourth nucleic acid probe, wherein the first nucleic acid probe is capable of hybridizing to a first target chromosomal DNA sequence, wherein the second nucleic acid probe is capable of hybridizing to a second target chromosomal DNA sequence, wherein the third nucleic acid probe is capable of hybridizing to a third target chromosomal DNA sequence, and wherein the fourth nucleic acid probe is capable of hybridizing to a fourth target chromosomal DNA sequence. In a further embodiment, the first probe is detectably labeled with a first reporter molecule, the second probe is detectably labeled with a second reporter molecule, the third probe is detectably labeled with a third reporter molecule, and the fourth probe is detectably labeled with a fourth reporter molecule. In one embodiment, the first reporter molecule is a first fluorochrome, the second reporter molecule is a second fluorochrome, the third reporter molecule is a third fluorochrome, and the fourth reporter molecule is a fourth fluorochrome. In another embodiment, one or more of the first reporter molecule, the second reporter molecule, the third reporter molecule, and the fourth reporter molecule is biotin. In certain embodiments of the method, wherein a reporter molecule is biotin, the method further comprises contacting the cell with streptavidin conjugated to a fluorochrome. In a specific embodiment, the cell is a somatic cell that remains morphologically intact in suspension, and in another specific embodiment, the somatic cell is a tumor cell. In another specific embodiment, the cell is a germ cell. In a certain embodiment, the germ cell is a sperm cell, wherein the sperm cell is a human sperm cell or a sperm cell from a non-human animal (a non-human sperm cell). In a certain embodiment, the chromosomal abnormality detected is sperm aneuploidy, wherein the aneuploidy detected is (a) the absence of a non-sex chromosome; (b) the presence of at least one extra copy of a non-sex chromosome; (c) the presence of more than one sex chromosome; or (d) the absence of sex chromosomes. In a particular embodiment, the cell is obtained from a biological sample, which is selected from semen, blood, bone marrow, lavage fluid, bladder washing, amniotic fluid, ascites, and a mucosal secretion. In another certain embodiment, a chromosomal abnormality is detected in a somatic cell, wherein the aneuploidy detected is (a) the presence of only one copy of a non-sex chromosome; (b) the presence of an extra copy of a non-sex chromosome (i.e., the presence of three or more copies of a non-sex chromosome); (c) the presence of only one sex chromosome; or (d) the presence of more than two sex chromosomes (i.e., three or more sex chromosomes).

In another embodiment, a method is provided for determining aneuploidy in a sperm cell, comprising (a) contacting the sperm cell with (i) a first nucleic acid probe that is capable of hybridizing to a target X chromosomal DNA sequence; (ii) a second nucleic acid probe that is capable of hybridizing to a target Y chromosomal DNA sequence; and (iii) a third nucleic acid probe that is capable of hybridizing to a target chromosomal DNA sequence of a non-sex chromosome, under conditions and for a time sufficient to permit interaction of chromosomal DNA in the sperm cell and the probe; and (b) detecting the hybridized first probe, the hybridized second probe, and the hybridized third probe by multispectral imaging of the sperm cell in flow. In certain embodiments, the sperm cell is a human sperm cell, and in other certain embodiments, the sperm cell is from a non-human animal (non-human sperm cell). In a specific embodiment, the first nucleic acid probe is detectably labeled with a first reporter molecule, the second nucleic acid probe is detectably labeled with a third reporter molecule, and the third nucleic acid probe is detectably labeled with a third reporter molecule. In a related embodiment, the first reporter molecule is a first fluorochrome, the second reporter molecule is a second fluorochrome, and the third reporter molecule is a third fluorochrome. In another embodiment, any one or more of the first reporter molecule, the second reporter molecule, and the third reporter molecule is biotin. In a further embodiment of the method wherein biotin is any one or more of the first, second, or third reporter molecule, the cell is contacted with streptavidin conjugated to a fluorochrome. In one embodiment, the aneuploidy detected is (a) the absence of a non-sex chromosome; (b) the presence of at least one extra copy of a non-sex chromosome; (c) the presence of more than one sex chromosome; or (d) the absence of sex chromosomes.

In another embodiment, a method is provided for identifying a sperm cell in a biological sample, comprising (a) directing brightfield and laser light at a cell; (b) obtaining a side scatter profile and brightfield image using a CCD detector; and (c) determining the spatial content of the side scatter profile and brightfield image, and therefrom identifying a sperm cell. In certain embodiments, relative movement exists between the cell and the detector. In a particular embodiment, the biological sample comprises a heterogeneous cell population. In a further embodiment, the method comprises multispectral imaging. In certain particular embodiments, multispectral imaging comprises (a) detecting in a first imaging channel a first nucleic acid probe that is hybridized to a first target chromosomal DNA sequence, wherein the first probe is attached to a first fluorochrome; and (b) detecting in a second imaging channel a second nucleic acid probe that is hybridized to a second target chromosomal DNA sequence, wherein the second probe is attached to a second fluorochrome. In further embodiments, the method comprising multispectral imaging comprises (a) determining a system mask area to user mask area ratio (first ratio) of the first fluorochrome detected in the first imaging channel; and (b) determining a system mask area to user mask area ratio of the second fluorochrome detected in the second imaging channel (second ratio). In a particular embodiment, the method further comprises plotting the first ratio against the second ratio on a bivariate scatter plot. In another embodiment for identifying a sperm cell in a biological sample further comprising multispectral imaging, multispectral imaging comprises (a) detecting in a first imaging channel a first nucleic acid probe that is hybridized to a first target chromosomal DNA sequence, wherein the first probe is attached to a first fluorochrome; and (b) detecting in a second imaging channel a second nucleic acid probe that is hybridized to a second target chromosomal DNA sequence, wherein the second probe is attached to a second fluorochrome; wherein multispectral imaging comprises (c) detecting in a third imaging channel a third nucleic acid probe that is hybridized to a third target chromosomal DNA sequence, wherein the third probe is attached to a first fluorochrome; and (b) detecting in a fourth imaging channel a fourth nucleic acid probe that is hybridized to a fourth target chromosomal DNA sequence, wherein the fourth probe is attached to a second fluorochrome. The a specific embodiment, the method further comprises further comprising (a) determining a system mask area to user mask area ratio (first ratio) of the first fluorochrome detected in the first imaging channel; (b) determining a system mask area to user mask area ratio of the second fluorochrome detected in the second imaging channel (second ratio); (c) determining a system mask area to user mask area ratio (third ratio) of the third fluorochrome detected in the third imaging channel; (d) determining a system mask area to user mask area ratio of the fourth fluorochrome detected in the fourth imaging channel (fourth ratio).

### BRIEF DESCRIPTION OF THE DRAWINGS

FIG. 1 shows a schematic representation of the ImageStream 100® multispectral imaging cytometer.

FIGS. 2A-2C show aggregates of sperm (A) stained with a chromosome 8 specific Spectrum Green labeled probe (the diffuse background shows the sperm clumps), (B) under brightfield imagery, and (C) stained with nuclear stain 7-amino actinomycin D (7-AAD).

FIG. 3 shows four collaged images from a fluorescent microscope showing individual human sperm without aggregates. Cells were stained with a Cy 3-labeled probe specific for the Y chromosome.

FIGS. 4A and 4B show fluorescent microscope imagery of human sperm after a completed procedure that included sonication, resulting in the lack of aggregates. (A) shows sperm labeled with the nuclear dye DRAQ5, and (B) shows chromosome 8 probe labeled with Alexa Fluor 488.

FIG. 5 represents a fluorescent microscope image of a Jurkat cell analyzed by FISH-IS. The green FISH dots represent Alexa Fluor 488 amplification of a fluorescein labeled chromosome 8 probe. The red FISH spot corresponds to the Cy3 labeled chromosome Y probe.

FIG. 6A depicts the first 11 of 20,000 separate Jurkat cells imaged using ImageStream. FIG. 6B represents the image of eleven cells of a gated in-focus population. FIG. 6C presents eleven cells for which a gate was determined for the best in-focus FISH spots. In each figure, from left to right, the multimode channels correspond to (i) darkfield or side scatter channel (400-470 nm), (ii) Alexa 488 channel corresponding to the labeled 8 chromosome (500-560 nm), (iii) Cy3 channel corresponding to the labeled Y chromosome (560-595 nm), and (iv) brightfield (595-660 nm).

FIG. 7 shows a fluorescent microscope image of a Jurkat cell subjected to FISH-IS. The green FISH dots represent Alexa Fluor 488 amplification of the fluorescein labeled chromosome 8 probe. The red FISH spot corresponds to the Cy3 labeled chromosome Y probe.

FIG. 8 shows an octagonal structuring element.

FIG. 9 shows an exemplary screen shot of the IDEAS™ Statistical Analysis Software.

FIG. 10 shows an interactive bivariate plot of the aspect ratio (brightfield) and the object area (brightfield) for collected data of human sperm cells subjected to FISH-IS and having chromosomes 8 and Y stained with different probes.

FIG. 11 shows a screen shot of the IDEA™ Statistical Analysis Software of sperm cells.

FIG. 12 shows brightfield and fluorescent (500-600 nm) images of murine sperm FISH-IS with chromosome 8 probe labeled with FITC.

### DETAILED DESCRIPTION

The present invention relates to the use of both photometric and morphometric features derived from multi-mode imagery of objects (e.g., cells) in flow to discriminate cell features, such as chromosomal abnormalities, in heterogeneous populations of cells, including non-adherent and adherent cell types. A surprising result described herein is the ability to discriminate between different cell states, such as distinguishing and identifying aneuploid cells from normal diploid cells and normal haploid cells such as sperm cells, by using multispectral imaging. Described in more detail below are methods for preparing cells typically difficult to prepare for in situ hybridization of a cell in suspension or flow, such as cells that tend to aggregate or have condensed chromosomes (for example, sperm cells). The methods comprise comprehensive multispectral imaging to provide morphometric and photometric features that allow, for example, the identification of chromosomes and chromosomal abnormalities not feasible with standard microscopy and conventional flow cytometry.

In the present description, any concentration range, percentage range, ratio range, or integer range is to be understood to include the value of any integer within the recited range and, when appropriate, fractions thereof (such as one tenth and one hundredth of an integer, etc.), unless otherwise indicated. As used herein, the term “about” means ±15%. As used herein, the use of an indefinite article, such as “a” or “an,” should be understood to refer to the singular and the plural of a noun or noun phrase (i.e., meaning “one or more” of the enumerated elements or components). The use of the alternative (e.g., “or”) should be understood to mean either one, both, or any combination thereof of the alternatives.

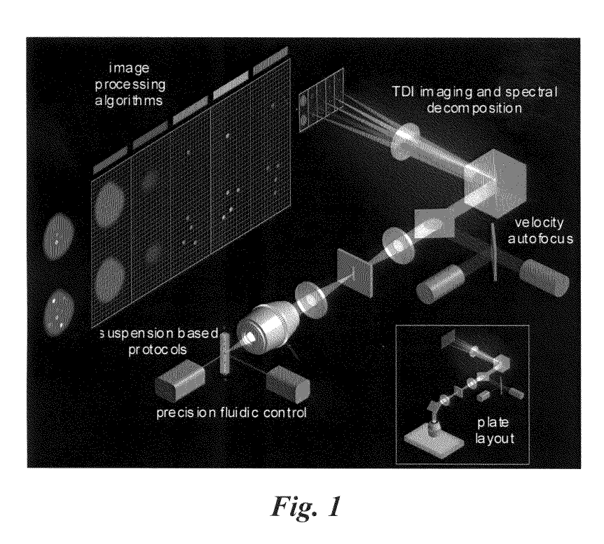

By way of background, methodologies for simultaneous high speed multispectral imaging in brightfield, darkfield, and four channels of fluorescence of cells in flow were recently developed (see, e.g., U.S. Pat. Nos. 6,211,955 and 6,249,341). FIG. 1 illustrates an exemplary imaging system (e.g., the ImageStream® platform). Cells are hydrodynamically focused into a core stream and orthogonally illuminated for both darkfield and fluorescence imaging. The cells are simultaneously trans-illuminated via a spectrally-limited source (e.g., filtered white light or a light emitting diode) for brightfield imaging. Light is collected from the cells with an imaging objective lens and is projected on a charge-coupled detector (CCD). The optical system has a numeric aperture of 0.75 and the CCD pixel size in object space is 0.5 microns square, allowing high resolution imaging at event rates of approximately 100 cells per second. Each pixel is digitized with 10 bits of intensity resolution, providing a minimum dynamic range of three decades per pixel. In practice, the spread of signals over multiple pixels results in an effective dynamic range that typically exceeds four decades per image. Additionally, the sensitivity of the CCD can be independently controlled for each multispectral image, resulting in a total of approximately six decades of dynamic range across all the images associated with an object.

Prior to projection on the CCD, the light is passed through a spectral decomposition optical system that directs different spectral bands to different lateral positions across the detector (see, e.g., U.S. Pat. No. 6,249,341). With this technique, an image is optically decomposed into a set of 6 sub-images, each corresponding to a different color component and spatially isolated from the remaining sub-images. This process allows for identification and quantitation of signals within the cell by physically separating on the detector signals that may originate from overlapping regions of the cell. Spectral decomposition also allows multimode imaging: the simultaneous detection of brightfield, darkfield, and multiple colors of fluorescence. This is exemplified in FIG. 1, which shows a red brightfield illumination source and the associated transmitted light images in the red detector channel adjacent to fluorescent and scattered light images in the other spectral channels. The process of spectral decomposition occurs during the image formation process rather than via digital image processing of a conventional composite image.

The CCD may be operated using a technique called time-delay-integration (TDI), a specialized detector readout mode that preserves sensitivity and image quality even with fast relative movement between the detector and the objects being imaged. As with any CCD, image photons are converted to photocharges in an array of pixels. However, in TDI operation the photocharges are continuously shifted from pixel to pixel down the detector, parallel to the axis of flow. If the photocharge shift rate is synchronized with the velocity of the flowing cell's image, the effect is similar to physically panning a camera: image streaking is avoided despite signal integration times that are orders of magnitude longer than in conventional flow cytometry. For example, an instrument may operate at a continuous data rate of approximately 30 megapixels per second and integrate signals from each object for 10 milliseconds, allowing the detection of even faint fluorescent probes within cell images that are acquired at high-speed. Careful attention to pump and fluidic system design to achieve highly laminar, non-pulsatile flow eliminates any cell rotation or lateral translation on the time scale of the imaging process (see, e.g., U.S. Pat. No. 6,532,061).

A real-time algorithm analyzes every pixel read from the CCD to detect the presence of object images and calculate a number of basic morphometric and photometric features, which can be used as criteria for data storage. Data files encompassing 10,000-20,000 cells are typically about 100 MB in size and, therefore, can be stored and analyzed using standard personal computers. The TDI readout process operates continuously without any “dead time,” which means every cell can be imaged and the coincidental imaging of two or more cells at a time, as depicted in FIG. 1, presents no barrier to data acquisition.

Such an imaging system can be employed to determine morphological, photometric, and spectral characteristics of cells and other objects by measuring optical signals, including light scatter, reflection, absorption, fluorescence, phosphorescence, luminescence, etc. As used herein, morphological parameters may be basic (e.g., nuclear shape) or may be complex (e.g., identifying cytoplasm size as the difference between cell size and nuclear size). For example, morphological parameters may include nuclear area, perimeter, texture or spatial frequency content, centroid position, shape (i.e., round, elliptical, barbell-shaped, etc.), volume, and ratios of any of these parameters. Morphological parameters may also include cytoplasm size, texture or spatial frequency content, volume and the like, of cells. As used herein, photometric measurements with the aforementioned imaging system can enable the determination of nuclear optical density, cytoplasm optical density, background optical density, and the ratios of any of these values. An object being imaged can be stimulated into fluorescence or phosphorescence to emit light, or may be luminescent wherein light is produced without stimulation. In each case, the light from the object may be imaged on a TDI detector of the imaging system to determine the presence and amplitude of the emitted light, the number of discrete positions in a cell or other object from which the light signal(s) originate(s), the relative placement of the signal sources, and the color (wavelength or waveband) of the light emitted at each position in the object.

Methods for performing fluorescent in situ hybridization in suspension (FISH-IS) on whole cells have been developed (see, e.g., U.S. Patent Application No. 2003/0104439), which differs from previously published methods that rely on the isolation of nuclei for performing FISH in suspended samples (van Dekken et al., Cytometry 11:153, 1990); Trask et al., Human Genetics 78:251, 1988; Arkesteijn et al., Cytometry 19:353, 1995; Wyrobek et al., Mol. Repro. Dev. 27:200, 1990; Shi and Martin, Cytogen. Cell Genetics 90:219, 2000). The adaptation of FISH to a whole cell suspension protocol combined with multispectral flow imaging facilitates high throughput analysis of cytogenetic features in cells by, for example: (1) simplifying sample preparation and handling through the elimination of slide preparations; (2) preserving the morphology of cells, such as sperm cells, and increasing the efficiency and uniformity of hybridization of probe cocktails by maintaining the cells in a fully suspended state; (3) increasing the fraction of in-focus probes by hydrodynamically focusing the cells with micron-scale accuracy; (4) speeding the analysis and increasing the spectral resolution of probes by simultaneous fluorescence imaging in as many as four colors; and (5) potentially resolving spatially overlapping probes by stereoscopic imaging.

Although the recently developed FISH-IS protocols are useful for many cells, manipulation of cells with, for example, condensed DNA (such as sperm cells) is challenging given the difficulties associated with the clumping of such cells. For example, chromosomes in sperm cells are compacted to a greater degree than the chromosomes of somatic cells. The basic proteins in sperm are protamines (instead of histones), which are partly responsible for this tight chromosomal packing. Unlike the nucleosomal histones, protamines contain disulfide bonds that need to be reduced for both in vivo fertilization and in vitro FISH (Perreault et al., Dev. Biol. 101:160, 1984).

By way of background, fixation procedures for cells undergoing analysis in flow cytometry are typically different than procedures for analyzing cells on microscope slides. Aldehydes that cross-link proteins to each other and to DNA are used for fixation, and permeabilization is generally achieved using a detergent. Flow procedures are intended to analyze cells that remain intact, do not clump, and remain permeable to staining reagents. Procedures used for preparing cells for FISH, which cells are to then be analyzed by flow cytometry, are especially challenging because cells are fixed and then exposed to an organic solvent at high temperatures for long periods of time during hybridization. The choice of probes and reporter molecules when analyzing cells in flow is also more limited. Certain probes that target chromosomal DNA, which is within the nucleus, may not penetrate the nuclear membrane well. In certain circumstances, some detergents that effectively permeabilize the plasma membrane do not permeabilize the nuclear membrane. Also, entry of certain fluorochromes, which may be used as reporter molecules attached to the probes, into the nucleus is less efficient (because of the fluorochrome mass). Fixation, permeabilization, and hybridization procedures may vary for different cell types. For example, preparation of sperm, particularly human sperm, for FISH-IS, which involves many steps, may lead to clumping of cells and loss of cells. Procedures described herein for preparation of cells with highly condensed chromosomes provide good yields of sperm cells in single-cell suspensions for use in FISH-IS.

The present disclosure provides methods of using both photometric and morphometric features derived from multi-mode imagery of objects in flow. Such methods can be employed for analyzing cells to determine one or more cell types, states of activation or differentiation, and cell features, in heterogeneous populations of cells when the cells are entrained in a fluid flowing through an imaging system. These exemplary methods may be used for imaging and distinguishing other moving objects that have identifiable photometric and morphometric features. As used herein, gating refers to a subset of data relating to photometric or morphometric imaging. For example, a gate may be a numerical or graphical boundary of a subset of data that can be used to define the characteristics of particles, objects, or cells to be further analyzed. Herein, gates are defined, for example, as a plot boundary that encompasses “in focus” cells, or sperm cells with tails, or sperm cells without tails, or cells other than sperm cells, or sperm cell aggregates, or cell debris. Further, backgating may be a subset of the subset data. For example, a forward scatter versus a side scatter plot in combination with a histogram from an additional marker may be used to backgate a subset of cells within the initial subset of cells.

In using an imaging system as described herein, a separate light source is not required to produce an image of the object (cell), if the object is luminescent (i.e., if the object produces light). However, many of the applications of an imaging system as described herein may require that one or more light sources be used to provide light that is incident on the object being imaged. A person having ordinary skill in the art appreciates that the location of the light sources substantially affects the interaction of the incident light with the object and thus determines the information that can be obtained from the images on a TDI detector.

In addition to imaging an object with the light that is incident on it, a light source can also be used to stimulate emission of light from the object. For example, a cell having been contacted with a probe conjugated to a fluorochrome (e.g., such as those described herein, including FITC, PE, AF488, GFP, Cy3, PE-Cy5, PerCP, and AF610-PE) will fluoresce when excited by light, producing a corresponding characteristic emission spectra from any excited fluorochrome probe that can be imaged on a TDI detector. Light sources may alternatively be used for causing the excitation of fluorochrome probes on an object, enabling a TDI detector to image fluorescent spots produced by the probes on the TDI detector at different locations as a result of the spectral dispersion of the light from the object that is provided by prism. The disposition of these fluorescent spots on the TDI detector surface will depend upon their emission spectra and their location in the object.

Each light source may produce light that can either be coherent, non-coherent, broadband, or narrowband light, depending upon the application of the imaging system desired. Thus, a tungsten filament light source can be used for applications in which a narrowband light source is not required. For applications such as stimulating the emission of fluorescence from probes, narrowband laser light is preferred, since it also enables a spectrally decomposed, non-distorted image of the object to be produced from light scattered by the object. This scattered light image will be separately resolved from the fluorescent spots produced on a TDI detector, so long as the emission spectra of any of the spots are at different wavelengths than the wavelength of the laser light. The light source can be either of the continuous wave (CW) or pulsed type, preferably a pulsed laser. If a pulsed type illumination source is employed, the extended integration period associated with TDI detection allows the integration of signal from multiple pulses. Furthermore, it is not necessary for the light to be pulsed in synchronization with the TDI detector.

According to the embodiments described herein, relative movement exists between the object being imaged and the imaging system. In certain embodiments, the object moves rather than the imaging system. In other embodiments, the object may remain stationary and the imaging system moves relative to it. As a further alternative, both the imaging system and the object may be in motion, and the movements of each may be in different directions and/or at different rates.

In one embodiment, a method is provided for identifying a sperm cell in a biological sample, comprising directing brightfield and laser light at a cell; obtaining a side scatter profile and brightfield image using a CCD detector; and determining the spatial content of the side scatter profile and brightfield image to determine the presence of a sperm cell in the biological sample. In certain embodiments, the spatial content that is analyzed is that of the cell nucleus. In other embodiments, multispectral imaging as described herein may be used to detect a sperm cell in a biological sample. A probe that detects a specific marker, such as a nuclear marker, or that detects a specific target chromosomal DNA sequence may be contacted with the cells in the biological sample as described herein. Incident light is directed at the marked cell, and a detector obtains an image of the cell. A sperm cell may be identified by using the nuclear marker image in combination with the spatial content of the cell image.

### Methods for Detecting Chromosomes and Detecting Chromosomal Abnormalities

Provided herein are methods using multispectral imaging of a cell in flow for detecting one or more chromosomes in a cell. Multispectral imaging of a cell in flow is also referred to herein as imaging flow cytometry. In one embodiment, the method comprises contacting a cell (combining, mixing, adding together, or otherwise introducing a probe to or into a cell) with a nucleic acid probe that is capable of hybridizing to a target chromosomal DNA sequence, under conditions and for a time sufficient to permit interaction of the target chromosomal DNA in the cell and the probe, and then detecting the hybridized probe by multispectral imaging of the cell in flow. In a particular embodiment, the cell is a sperm cell.

In another embodiment, a method is provided for determining whether a chromosomal abnormality exists in a cell. An abnormality of one or more chromosomes may be detected by contacting a cell (combining, mixing, adding together, or otherwise introducing a probe to or into a cell) with a nucleic acid probe that is capable of hybridizing to a target chromosomal DNA sequence, under conditions and for a time sufficient to permit interaction of the chromosomal DNA and the probe, and comparing the multispectral imaging of the cell in flow to the multispectral imaging of a chromosomally normal cell in flow, and thereby determining the presence of a chromosomal abnormality in the cell.

The practice of including appropriate controls in a method and establishing whether the results provide statistically significant or biologically significant observations and data when compared with controls is familiar to a person skilled in the art. The comparison between a cell that is being tested in the methods described herein for determining the presence of an abnormality in the cell with a cell that is known to contain normal chromosomes and the normal number of chromosomes for that cell (i.e., a diploid number for a somatic cell; a haploid number for a germ cell) can be accomplished at a time proximal to the time when the cell to be tested is analyzed. Alternatively, the comparison can be made on the basis of data and information obtained prior to or after the time when the method is performed for determining the presence of a chromosome and/or a chromosomally abnormality in a cell.

A chromosome and/or an abnormality of a chromosome may be detected in a somatic cell or in a germ cell. The methods described herein are useful for analyzing cells that maintain morphological integrity and remain intact when the cells are in suspension. A somatic cell includes a tumor cell, a stem cell, or any other cell that may be obtained, for example, from a biological sample and that is, or can be, established or maintained in suspension.

In certain embodiments, the cell is a germ cell such as a sperm cell. The sperm cell may be from any animal whose reproduction involves fertilization of an ovum by sperm, including a human or a non-human animal. Non-human animals include, for example, mammals (a non-human primate, a rodent (e.g., mouse, rat, rabbit), a dog, cat, goat, sheep, horse, bovine, or pig), fish, birds, etc. Collected sperm may be frozen, stored, and thawed according to standard methods that preserve the morphological integrity of the cells or may be collected by surgical sperm retrieval for collecting sperm from the vas deferens, epididymis or testis. Collection, freezing, and thawing of a biological sample containing sperm can be performed according to methods familiar to persons skilled in the art.

A somatic cell or germ cell may be obtained from a biological sample that comprises cells from a subject or biological source. In certain embodiments the biological sample is a biological fluid, which is typically a liquid at physiological temperatures and may include naturally occurring fluids present in, withdrawn from, expressed or otherwise extracted from a subject or biological source. Alternatively, the biological fluid may be stored frozen and later thawed according to standard methods for preserving morphological and physiological integrity of a cell. Examples of biological fluids include semen, blood, serum and serosal fluids, plasma, lymph, urine, cerebrospinal fluid, saliva, amniotic fluid, bone marrow, mucosal secretions of the secretory tissues and organs, vaginal secretions, ascites fluids such as those associated with non-solid tumors, fluids of the pleural, ductal, nasal, pericardial, peritoneal, abdominal and other body cavities, and the like. Biological fluids may also include liquid solutions contacted with a subject or biological source, for example, cell and organ culture medium including cell or organ conditioned medium, lavage fluids, such as lung lavage, ductal lavage, bladder washings, and the like. The biological sample may contain a heterogeneous population of cells, that is, numerous different types of cells.

The subject or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines and the like. Primary cell cultures or culture-adapted cell lines may grow in suspension or may be adherent to plastic. Adherent cells may be harvested according to standard methods for use in the methods described herein. In certain embodiments, the subject or biological source may be suspected of having or being at risk for having a malignant condition, and in certain other embodiments, the subject or biological source may be known to be free of a risk or presence of such disease.

The multispectral imaging in flow method described herein may be used for differentiating (distinguishing) and identifying different cell types present in a biological sample. For example, the presence or identity of a sperm cell in a biological sample may be detected among different cell types according to the methods described herein.

Any of the non-sex chromosomes (numbered chromosomes) or any one of the sex-related chromosomes (X and Y chromosomes) may be detected in a cell using the methods described herein. The number of chromosomes (diploid number refers to the total number of chromosomes in a somatic cell; haploid refers to the number of chromosome pairs) is identical within a somatic cell (diploid) or a germ cell (haploid) of the same species. As used herein, a non-sex (numbered) chromosome refers to a chromosome that is not an X or a Y chromosome, and refers to a particular chromosome as numbered according to the conventional and well known numbering system used by persons skilled in the art. In a normal somatic cell, the non-sex chromosomes are present as pairs of chromosomes, and in a germ cell only one chromosome of a pair of chromosomes is present. In a normal female somatic cell, two X chromosomes are present, and in a normal male somatic cell, one X chromosome and one Y are present. An ovum (female germ cell) contains a single X chromosome; a normal sperm cell (male germ cell) contains either an X chromosome or a Y chromosome.

In one embodiment, the methods described herein are used for detecting a chromosomal abnormality, which includes aneuploidy; a chromosome translocation, inversion, or rearrangement; gene amplification; gene mutation; gene deletion; a point mutation; or other DNA sequence abnormalities or mutations. An example of a chromosome rearrangement in humans is the Philadelphia chromosome, which is an abnormally short chromosome 22 that rearranges with chromosome 9, and which is a hallmark of chronic myeloid leukemia.

Aneuploidy refers to a chromosomal state in which abnormal numbers or sets (or pairs) of chromosomes rather than the normal diploid number are found in the nucleus. For example, trisomy refers to the presence in a somatic cell of three chromosomes rather than the normal two chromosomes (diploid), and tetrasomy refers to the presence of four chromosomes. A partial trisomy occurs when a portion of an extra chromosome is attached to another chromosome. Monosomy is an aneuploidy characterized by the presence of a single chromosome rather than the normal diploid number. Partial monosomy occurs when a long arm or short arm of a chromosome is missing. Accordingly, for example, in a germ cell, such as a sperm cell, aneuploidy is indicated by the presence of at least one extra chromosome of a chromosome pair, such as two chromosomes of a chromosome set (contributing to trisomy), or by the lack of a particular chromosome (contributing to monsomy).

In one embodiment, a method is provided for determining aneuploidy in a somatic cell or in a sperm cell, using cells that are in suspension. The aneuploidy detected may be the absence of a non-sex chromosome (i.e., in a somatic cell only one of a pair of chromosomes is present and in a germ cell, the non-sex chromosome is absent) or may be the presence of at least one extra copy of a non-sex chromosome (i.e., in a somatic cell, 3, 4, or more copies of a particular non-sex (numbered chromosome) are present; in a germ cell, 2, 3, or more copies of a particular non-sex (numbered chromosome) are present). The presence of at least one extra copy of a non-sex chromosome may be detected by multispectral imaging of the cell in flow using a probe that is capable of hybridizing to a specific DNA sequence located on the non-sex (numbered) chromosome. For example, the presence of an extra copy of a non-sex chromosome (three or more copies in a somatic cell) may be detected. In a sperm cell, two or more copies (e.g., two or more of number 21, number 13, or number 18, or number 8) rather than the normal haploid number may be detected by contacting the sperm cell (combining, mixing, adding together, or otherwise introducing a probe to or into a cell) with a probe that is capable of hybridizing to a specific DNA sequence located on the non-sex (numbered) chromosome.

In another embodiment, the presence of aneuploidy may be determined that is a sex-chromosomal aneuploidy, which refers to an abnormal number of sex chromosomes. A probe that hybridizes to a specific DNA sequence located on the X chromosome and/or a probe that hybridizes to a specific DNA sequence located on the Y chromosome may be contacted with a cell (a sperm cell or a somatic cell). In a somatic cell, such an aneuploidy includes the presence of three X chromosomes; two X chromosomes and one Y chromosome; and two Y chromosomes and one X chromosome. In a germ cell, such as a sperm cell, a sperm aneuploidy includes the presence of more than one sex chromosome (two Y chromosomes, two X chromosomes, or one each of an X chromosome and a Y chromosome) and includes the absence of sex chromosomes. Thus upon fertilization the resulting somatic cell comprises XYY, XXX, XXY, and X0 aneuploidies, respectively.

The methods described herein for determining chromosomal abnormalities may include probes that are capable of hybridizing to a DNA sequence present in a sex chromosome and include probes that are capable of hybridizing to a DNA sequence that is present in a non-sex chromosome. By using multispectral imaging analysis of a cell in flow, for example, a probe specific for the X chromosome, a probe specific for the Y chromosome, and two additional probes, each specific for a different non-sex (numbered) chromosome may be included in a method for determining aneuploidy.

### Nucleic Acid Probes

A probe that is a nucleic acid probe includes a polynucleotide or an oligonucleotide, which may be a single-stranded DNA, double-stranded DNA, single-stranded RNA, double stranded RNA, a RNA-DNA hybrid, or a peptide nucleic acid (PNA). As described in greater detail herein, the probe can be directly labeled with a reporter molecule or can be labeled with a reporter molecule (e.g., biotin) that permits indirect detection of the probe to the specific chromosomal DNA. The length (number of nucleotides) of the probe can vary and can be adjusted to detect a single mismatch (e.g., a probe comprising 12-50 nucleotides) though probes with a greater number of nucleotides can be employed. Probes that are used in the methods described herein comprise nucleotide sequences that are complementary to and therefore specifically hybridize to a DNA sequence on a particular numbered chromosome or sex-chromosome. The nucleic acid probes with specificity for a specific target DNA sequence on a specific chromosome may be designed and prepared by methods well known in the molecular biology and chemistry arts or purchased commercially.

In certain embodiments, a probe may have one or more effector or reporter molecules attached (or conjugated) to it. A reporter molecule may be a detectable moiety or label such as an enzyme or other reporter molecule, including a dye, radionuclide, luminescent group, fluorescent group (fluorochrome), a quantum dot (a small device that contains a tiny drop of at least one electron and may contain thousands of electrons), or biotin, or the like. Other reporter molecules include chelated lanthanide series salts, especially Eu³⁺, chromophores, radioisotopes, chelating agents, colloidal gold, latex particles, and chemiluminescent agents.

A reporter molecule such as a fluorochrome may be attached directly (labeled or conjugated) to a probe that interacts with (binds to or hybridizes with) a target molecule (such as a nucleic acid) in a cell. Alternatively, interaction between a probe and the target molecule may be detected indirectly; for instance, a secondary reagent may be labeled with a fluorochrome. The secondary reagent may interact with the probe or the secondary reagent may interact with a molecule that interacts with the probe. For instance, a ligand, such as biotin, may be attached to the probe. The hybridized probe is then detected by contacting (combining, mixing, adding together) the cell containing the hybridized probe with avidin or streptavidin (i.e., a binding partner of biotin) that is conjugated to a fluorochrome. Such indirect detection methods, which may be used to amplify a signal, are familiar to persons skilled in the art and are described herein. Another example of an indirect method is a tyramide amplification system (TSA™), which may be used to increase resolution and/or amplify a signal (see, e.g., Perkin Elmer, Boston, Mass.; Molecular Probes, Invitrogen Life Technologies).

Exemplary fluorochromes that may be conjugated to a probe or reagent include but are not limited to fluorescein isothiocyanate (FITC); phycoerythrin (PE); Alexafluor 488 (AF488); cyanine 3 (Cy3); Alexafluor 546 (AF546); spectrum green (spec. green); green fluorescent protein (GFP); a syto green fluorochrome; 7-aminoactinomycin D (7AAD); peridinin chlorophyll protein (PerCP); and DRAQ-5™.

The standard ImageStream® instrument used for multispectral imaging of a cell has six multi-spectral imaging channels. One channel is assigned to brightfield and a second channel is assigned to dark field (laser scatter); the four remaining channels are available for detection of four distinct probes. For example, Channel 1 (470-500 nm), may be used for darkfield laser side scatter channel (488 SSC); Channel 2 (400-470 nm), used for brightfield; Channel 3 (500-560 nm), used for brightfield or a fluorochrome such as FITC, Alexa 488, GFP, Syto, or Spec.Green; Channel 4 (560-595 nm), used for brightfield or a fluorochrome such as PE, or Cy-3; Channel 5 (595-660 nm), used for brightfield or a fluorochrome such as 7-AAD, or Alexa610/PE; and Channel 6 (660-730 nm), used for brightfield or a fluorochrome such as PE-Cy5, Alexa680/PE, Alexa647/PE, PerCP, or Draq-5. The dyes listed for each channel represent a partial list of available 488 nm-excitable fluorescent dyes that can be detected in each channel.

### Preparation of Cells for Detection of Chromosomes and Chromosomal Abnormalities Using Multispectral Imaging of the Cell in Flow

As described in detail herein multispectral imaging of a cell in flow permits viewing and analysis of a morphologically intact cell. Conditions and techniques that preserve cell integrity are, therefore, optimized for multispectral imaging of the cell. In preparation for hybridization of a probe to a target chromosomal DNA sequence, cells are fixed and permeabilized. In addition, for germ cells, such as sperm cells, the cells are exposed to a reducing agent to decondense the chromosomal DNA so that it is accessible to the probe.

According to procedures described herein and with which skilled artisans are familiar, cells are treated or fixed with a fixative solution to stabilize cellular structures and organelles, such as the nucleus, and then are permeabilized to permit entry of a probe or probes into the cell and/or the cell nucleus. The fixative and permeabilization agents and solutions selected are those that minimize cell aggregation or cell clumping, which occurs with sperm cells.

An example of a fixative is an aldehyde. Aldehydes may fix cells, at least in part, by denaturation and chemical modification of proteins, that is, by covalent reaction with free amino groups of particular amino acids, such as lysine residues. The fixation may alter peptide chain antigens of intracellular proteins while the glycoantigens of the cell membrane glycocalix remain largely unaffected. Cells become rigid because protein cross-linking occurs and consequently the cells suspend well. Examples of aldehydes include formaldehyde, paraformaldehyde, and glutaraldehyde. For fixing sperm cells, the aldehyde is preferably a zero-length cross-linker, for example, a zero-length cross-linker formaldehyde, which may be obtained as a paraformaldehyde powder or as a liquid product (see, e.g., Fix and Perm Solution A®, Caltag Laboratories, Burlingame, Calif.). The zero-length cross-linker formaldehyde, such as paraformaldehyde, also may render the cell permeable to probes that are subsequently contacted (or exposed to) the cell.

Alternatively, an alcohol or acetone may be used as a fixative. The alcohol may be a short chain alcohol, such as ethanol or methanol. The alcohol may be used as a 100% solution or may be diluted. The alcohol or acetone may be mixed with water or with another aqueous solution such as acetic acid, for example, to form a methanol:acetic acid solution (e.g., 3:1 methanol:acetic acid) or an ethanol:acetic acid solution. When the presence of a chromosome and/or a chromosomal abnormality in a germ cell, such as a sperm cell, is being detected, the fixative is preferably a diluted alcohol containing solution and not a 100% alcohol solution. Carnoy's solution (3:1, methanol:acetic acid) is preferably used. Fixation in Carnoy's solution may be performed in two steps: for example, sperm cells may be treated with undiluted Carnoy's solution, followed by a second fixative treatment with a diluted Carnoy's solution (which may be 60%, 70%, 80% or 90%) Carnoy's. By using a diluted alcohol fixative solution, clumping of sperm cells is reduced and the yield of single sperm cells is improved.

Fixation of a cell, such as a sperm cell may include treating the cell in separate steps with a solution containing an alcohol and with an aldehyde. For example, sperm cells may be first exposed to a fixative solution comprising a diluted alcohol (such as Carnoy's solution), removing the cells from the alcohol-containing fixative, and then treating the sperm cells with a zero-length cross-linker formaldehyde fixative solution (e.g., paraformaldehyde or a solution thereof). Fixation may also comprise two treatments with a Carnoy's solution or a diluted Carnoy's solution followed by an aldehyde fixation step. For example, sperm cells may be treated first with undiluted Carnoy's solution, then treated with diluted Carnoy's solution (e.g., 30% Carnoy's), followed by treatment with paraformaldehyde (such as 1% paraformaldehyde).

When detecting a chromosome and/or a chromosomal abnormality in a germ cell, such as sperm cell, prior to fixing the cells, the germ cells are exposed to a solution that causes decondensation of the chromosomal DNA. Chromosomes in sperm cells are compacted to a greater degree than the chromosomes of somatic cells. The basic proteins in sperm are protamines (instead of histones), which are partly responsible for this tight chromosomal packing. Unlike the nucleosomal histones, protamines contain disulfide bonds that are reduced for in vitro FISH. Accordingly, sperm cells are treated with a reducing agent, such as dithiothreitol.

Clumping of sperm cells may be reduced by sonicating the sperm cells, which removes the tails from a majority of the sperm. Sonication may be performed prior to fixation, after fixation and prior to hybridization of the probe to the target chromosomal DNA sequence, or after hybridization. Sonicating the sperm after fixation can remove tails from approximately 95% of the sperm. Sonication prior to fixation is less effective in preventing or minimizing clumping of sperm cells, but does not deleteriously alter the morphological integrity of the cells.

### Hybridization

Hybridization of a nucleic acid probe with a specific chromosomal DNA target sequence is performed according to methods described herein and practiced in the art (thus, providing conditions and time sufficient to permit interaction of a probe and a target chromosomal DNA in a cell) (see, e.g., Examples and references cited herein). A hybridization buffer comprises the nucleic acid probe and a buffering agent, and additional components such that the buffer is suitable for permitting binding of a probe to its corresponding specific target chromosomal DNA in an intact cell. A hybridization buffer may also comprise a blocking agent to reduce non-specific binding. In addition, a hybridization buffer may contain a chaotropic agent (e.g., formamide) that lowers the melting temperature (Tm) of nucleic acid duplexes. Formamide present in the hybridization buffer and/or in the post-hybridization wash buffer may contribute to denaturing of surface proteins of a cell. If the cell is a sperm cell, the exposure of the cells to formamide may contribute to clumping of the sperm. Clumping may be reduced by including a non-ionic detergent (e.g., Triton® and Tween®) or a glycoside (e.g. saponin) in the hybridization and/or wash buffer.

### Spot Feature Analysis

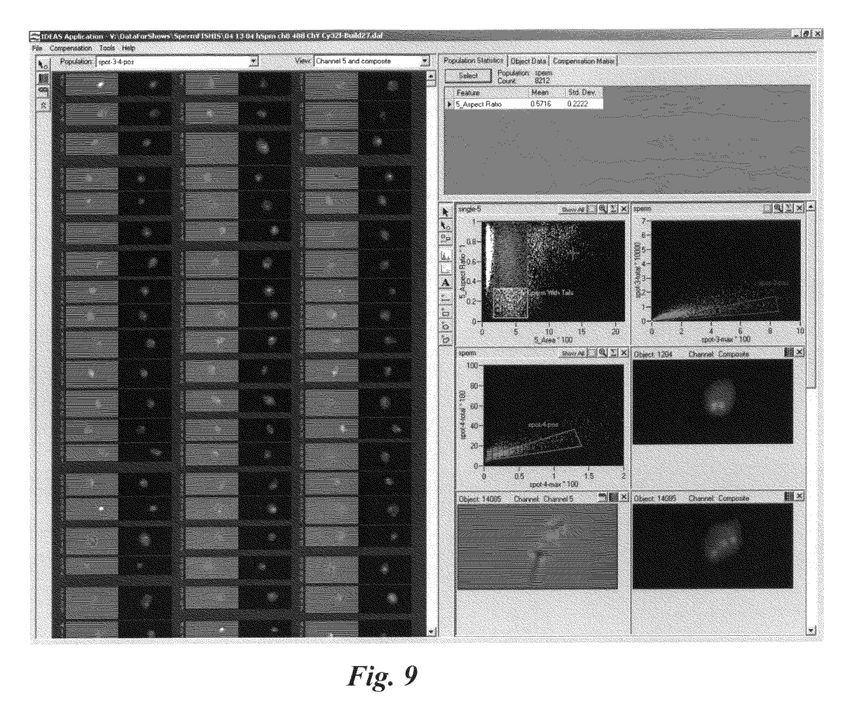

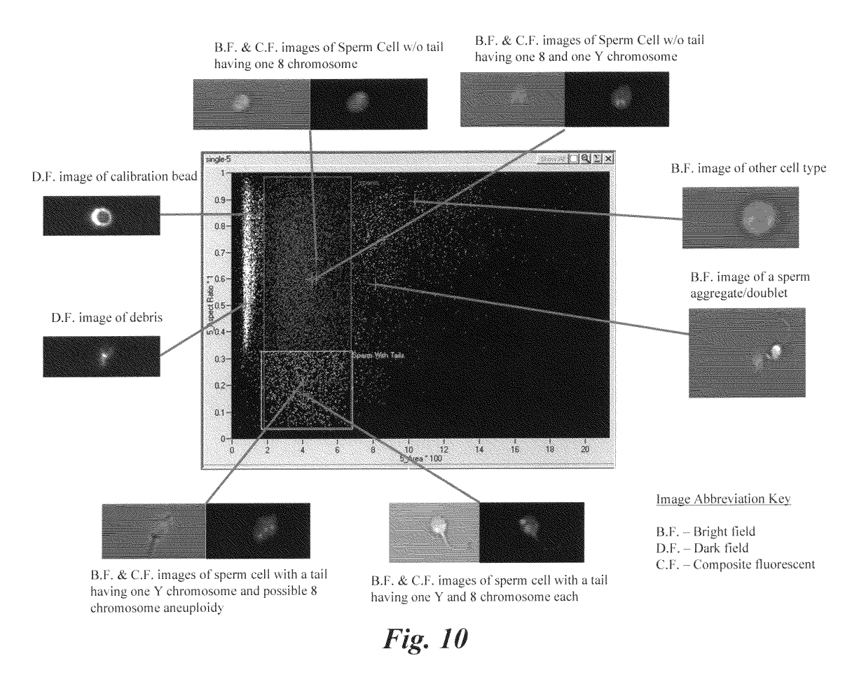

An exemplary screen shot of the IDEAS™ Statistical Analysis Software is shown in FIG. 9. Within the workspace screen area (right hand side), the aspect ratio of the brightfield image (y-axis) is plotted against the area of the brightfield image of the object x-axis). The area inscribed by the red rectangle was found to best describe the imaged objects that were determined to be sperm cells. Within this population, the yellow rectangle representing a lower aspect ratio was found to be a good classifier for sperm cells having tails attached. The group of objects to the left of the sperm gate was composed of both debris and calibration beads that are in flow with the sperm cells. Additionally, the outlier population to the right was primarily sperm aggregates and other cell types. FIG. 10 graphically illustrates this scatter plot and illustrates the power of the IDEAS™ and ImageStream® platform where one can click on the scatter plot data point and view the collected multispectral imagery.

The second scatter plot shown in the workspace of FIG. 9 shows the Total Spot Intensity (y-axis) versus the Spot area x-axis) for the Cy-3 imaging channel (560-595 nm). The area inside the purple inscribed gate was determined to be in-focus sperm cells having FISH spots for the Cy-3 labeled Y-Chromosome. Additionally, a similar scatter plot was created and illustrated in FIG. 10 using the Alexa 488 imaging channel (500-560 nm) for the Alexa 488 labeled Chromosome 8 probe. The following provides a description of the Spot intensity features used above.

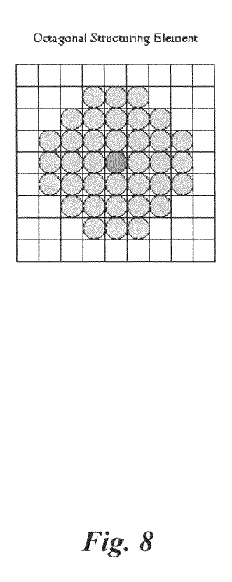

A set of features to quantify the presence of small bright sources in images is provided herein as a useful first step toward identifying cells with one or more FISH spots present. The images are processed according to the following algorithm. First, an erosion is performed, followed by a corresponding dilation. The resulting image is subtracted from the original, and the total and peak intensities remaining are computed. The erosion and dilation are performed with an octagonal structuring element, pictured in FIG. 8.

The squares in the structuring element represent the grid of pixels, while the gray circles highlight those pixels belonging to the structuring element. The pixels on the ImageStream instrument are approximately 0.5 microns across; accordingly, the structuring element, at 7 pixels, is 3.5 microns. To perform the erosion, the structuring element is (figuratively) placed with its darkened center circle over each pixel of the source image, and the intensity of the center pixel in the output image is set to the minimum intensity of all those pixels in the source image covered by circles. The dilation proceeds in the same way, except the maximum intensity is used instead of the minimum.

The net effect of the combination of erosion and dilation, called an opening, is that localized bright sources, which are less wide than the structuring element, are removed from the image while wider sources and local minima remain. When the opened image is subtracted from the original image, only the bright regions narrower than the structuring element remain. The peak and total intensity of these regions reflect the presence of small bright sources in the imagery.

### User Mask Analysis

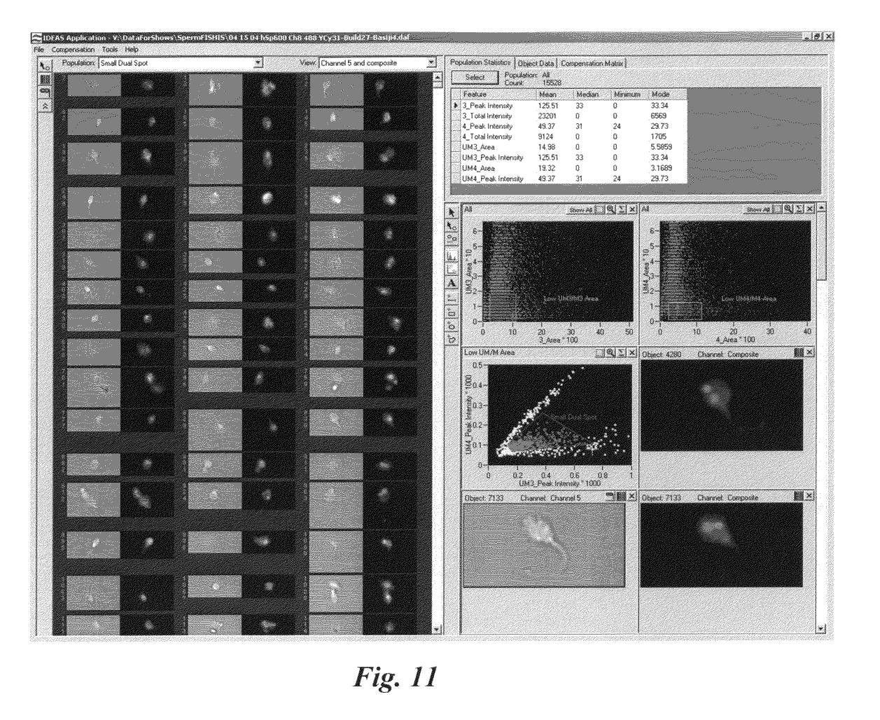

A second analysis technique has been applied to the human sperm FISH-IS ImageStream data file. This file was collected after the first method “Spot Feature Analysis.” As a result, the real time image segmentation algorithm was altered, which excluded calibration bead imagery and cellular debris. The resulting collected imagery had a much higher percentage of sperm cells included. FIG. 11 illustrates the Workspace screen area for the IDEAS Statistical and Analysis Software.

A feasibility classifier was developed to identify doubly-labeled cells that used differences in setting of object detection. The system mask (M) operates with a low threshold (sensitivity), whereas the user mask (UM) can be set to recognize only strong signals. This consists of the following: (1) set a user mask of 25% for each of the FISH colors (UM3 and UM4, for example); (2) create two new simple features that are the areas of the user masks; (3) create two new complex features that are the ratios of the system mask to user mask areas (M3/UM3 Area, M4/UM4 Area); (4) plot the complex features against each other on a bivariate scatter plot; and (5) define a population as those cells that have both high M3/UM3 Area and M4/UM4 Area. Cells with small FISH spots will have a small user mask and therefore a high system mask to user mask area ratio.

A system mask area to user mask area ratio may be determined for each channel in which imagery is performed; therefore, cells that are labeled with four different fluorochromes can be analyzed. Thus FISH spots can be identified and quantified by using multispectral imaging and determining the system mask to user mask area ratios.

For example, a feasibility classifier was developed to identify doubly-labeled sperm (chromosomes Y and 8) that used differences in setting of object detection. This consisted of the following: (1) setting a user mask of 25% for each of the FISH colors (UM3 and UM4, for example); (2) creating two simple features that were the areas of the user masks; (3) creating two complex features that were the ratios of the system mask to user mask areas (M3/UM3 Area, M4/UM4 Area); (4) plotting the complex features against each other on a bivariate scatter plot; and (5) defining a population as those cells that have both high M3/UM3 Area and M4/UM4 Area. When cells were exposed to (contacted) a chromosome 8 specific probe and a chromosome Y specific probe, the image gallery illustrated that cells within this classifier had positive FISH spots for chromosome 8 and Y (see FIG. 11).

In another embodiment, a sperm cell in a biological sample is identified by a method that comprises directing brightfield and laser light at a cell; obtaining a side scatter profile and brightfield image using a CCD detector; and determining the spatial content of the side scatter profile and brightfield image to determine the presence of a sperm cell in the biological sample. As described herein, the spatial content that is analyzed is that of the cell nucleus. The method may further comprise multispectral imaging, including determining the system mask area and user mask area for each FISH color (fluorochrome). Two, three, or four different nucleic acid probes that specifically bind to different target chromosomal DNA sequence may be contacted with a cell, such as a sperm cell. The probes are directly or indirectly labeling with a reporter molecule, such as a fluorochrome, and each hybridized probe is detected in an imaging channel.

The following Examples are offered by way of illustration and not by way of limitation.

### EXAMPLES

### Example 1

### Evaluation of Fixatives for Sperm Cells

Preservation of cell integrity when analyzing cells by FISH-IS provides optimal results. This Example describes evaluation of various fixatives, including cross-linking fixatives and denaturing fixatives. The cross-linking fixative tested was the zero-length cross-linker formaldehyde, which may be obtained as paraformaldehyde powder or as a liquid product from various commercial vendors (e.g., Fix and Perm® Solution A, Caltag Laboratories, Burlingame, Calif.).

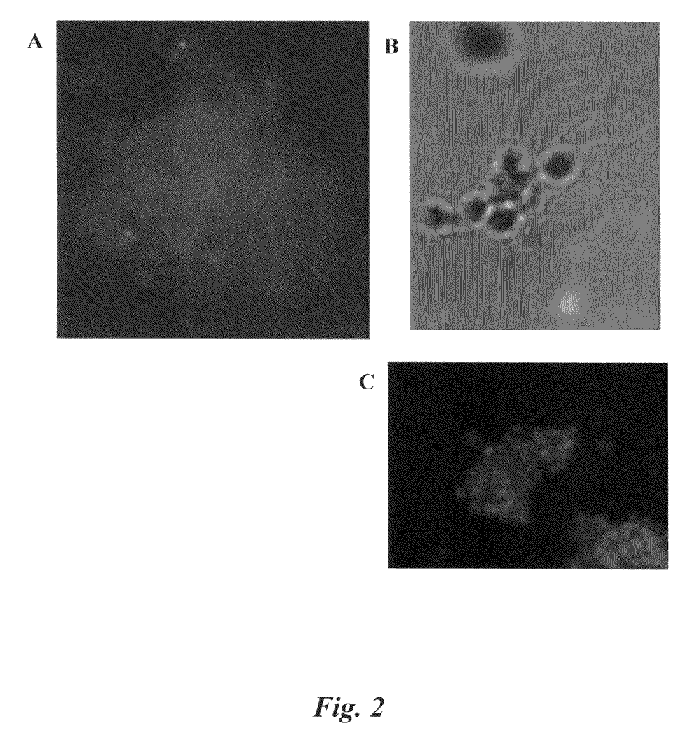

Several experiments were performed with variations of the procedure for preparing sperm cells for hybridization with a probe. Briefly, the basic procedure included treating sperm cells with dithiothreitol (DTT), followed by centrifugation; treating the cells with lithium diiodosalicylate; removing the cells from the lithium diiodosalicylate by centrifugation; fixing the cells; centrifuging and washing the cells, and then performing a hybridization reaction. Despite varying the time period for fixation, speed of centrifugation, method of addition of lithium diiodosalicylate, and whether fixation was performed before or after dithiothreitol treatment, no procedure gave satisfactory yields of unaggregated single sperm cells. Sperm tended to clump together forming some large aggregates that were visible to the naked eye as well as forming small clumps that were visible under the microscope (FIG. 2). These clumps led to very poor yields of sperm. Losses varied and occurred at each of the several steps. In particular experiments, as many as 90% of the cells were lost in the steps up to hybridization. The overnight hybridization step itself often led to large losses of material and severe clumping of the remaining material. Large, dense clumps of sperm were difficult to analyze under the microscope; however, some small aggregates appeared to have the sperm tails wrapped around each other while others had head to head contacts. Although, many early attempts at sperm in-suspension preparations and hybridizations all resulted in poor yields, the procedure for preparing sperm cells for FISH-IS was reevaluated as a whole in an attempt to identify single ineffective steps, with a goal of fewer steps in total, less time, and fewer centrifugation steps.

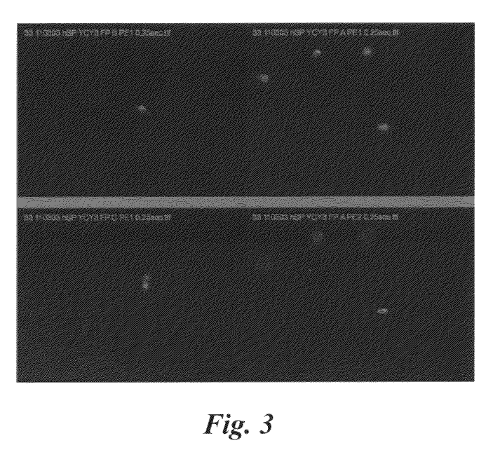

Subsequent experiments eliminated both the lithium diiodosalicylate step and the centrifugation after the dithiothreitol step. Various formaldehyde fixatives were compared as well. Results showed a better yield in the abbreviated procedure as illustrated in FIG. 3. In addition, the recovered cells had very few clumps. On the basis of these results, the fixative chosen for subsequence experiments was formaldehyde (e.g., Fix and Perm® Solution A, Caltag).

Several alcohol-denaturing fixatives were also evaluated including methanol, ethanol, and Camoy's solution (methanol/acetic acid). Initial attempts with 100% alcohol fixatives led to poor recovery of cells and clumping of cells. However, the yield of the single sperm cells was improved as many as two-fold when lower concentration of alcohol fixatives were used. See Examples 5, 7, and 9.

The loss of cells during the process may be due to disintegration of the cells and/or to the presence of free chromosomal DNA that may increase the clumping of cells. To resolve this problem, sperm cells were first fixed in a low concentration of alcohol fixatives and then fixed in low concentration of paraformaldehyde for short period of time. The total yield of single sperm cells was increased as many as three-fold compared with data obtained from experiments in which higher concentrations of paraformaldehyde were used and the cells were exposed for longer periods of time.

### Example 2

### Preparation of Sperm Cells in Suspension without Clumping

The yield and clumping problems associated with the hybridization step were subsequently examined. Whether losses were due to the high temperatures used during hybridization or to the use of formamide were investigated. Mock hybridizations were performed in which the hybridization mix was incubated at 37° C. overnight. The results showed that the losses were not dependent on temperature, but were likely caused by exposing cells to formamide.

Because formamide may denature surface proteins, the addition of a detergent during hybridization was examined to determine if the presence of a detergent would minimize cell loss due to clumping. In theory, cells should be relatively stable to detergent once they are fixed. Non-ionic detergents (Triton® and Tween®) and glycosides (e.g. saponin) most effectively minimized loss of sperm cells due to clumping of the cells. In certain embodiments, 1% Saponin was used.

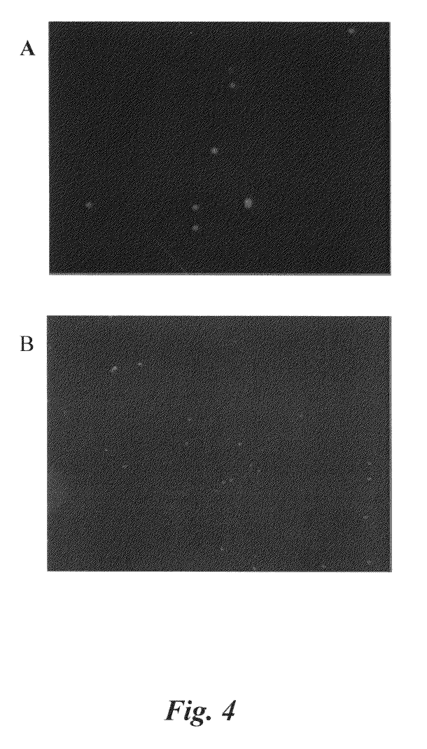

In a further attempt to decrease sperm clumping, sonication using an Aquasonic 75HT sonifier (VWR) after fixation was performed. An exposure time of 10 minutes was sufficient to remove tails from about 94% of the sperm. Sonication before fixation was less effective in removing tails, but seemed not to harm the sperm cells. Also, the few small clumps that sometimes appeared post-hybridization could be disrupted by another round of sonication (FIG. 4).

### Example 3

### Efficient Fish-IS Procedure for Sperm Cells

The standard ImageStream® instrument uses six multi-spectral imaging channels as follows: Channel 1, 470-500 nm, darkfield laser side scatter channel (488 SSC); Channel 2, 400-470 nm, used for brightfield; Channel 3, 500-560 nm, used for brightfield, FITC, Alexa 488, GFP, Syto, or Spec.Green; Channel 4, 560-595 nm, used for brightfield, PE, or Cy-3; Channel 5, 595-660 nm, used for brightfield, 7-AAD, or Alexa610/PE; and Channel 6, 660-730 nm, used for brightfield, PE-Cy5, Alexa680/PE, Alexa647/PE, PerCP, or Draq-5. The dyes listed for each channel represents a partial list of available 488 nm-excitable fluorescent dyes that can be detected in each channel. Because one channel is assigned to brightfield and a second channel is assigned to dark field (laser scatter), four channels remain available for distinct probes. See Table 1.

TABLE 1
ImageStream ® Imagine Channels

| 1 | Channel 1 | Channel 2 | Channel 3 | Channel 4 | Channel 5 | Channel 6 |
|---|---|---|---|---|---|---|
| 2 | 470-500 nm | 400-470 nm | 500-560 nm | 560-595 nm | 595-660 nm | 660-730 nm |
| 3 | 488 SSC | Bright Field | Bright Field | Bright Field | Bright Field | Bright Field |
| 4 | | | FITC | PE | 7AAD | PE-Cy5 |
| 5 | | | Alexa 488 | Cy-3 | Alexa610/PE | Alexa680/PE |
| 6 | | | GFP | | | Alexa647/PE |
| 7 | | | Syto | | | PerCP |
| 8 | | | Spec.Green | | | Draq-5 |

The following procedure was developed for detecting the presence of three chromosomes, 8, X, and Y, in human and murine sperm cells. Probes for the human chromosomes were obtained from Cambio (Cambridge, UK). Each of the probes is directed to (specific for) a satellite DNA sequence. The chromosome 8 probe was labeled with fluorescein isothiocyanate (FITC) and the Y chromosome probe was derivatized with the cyanine 3 (Cy3) dye. The peak emission for fluorescein is in Channel 3 of the ImageStream, while the peak emission for Cy3 is in Channel 4. The probe for the X chromosome was conjugated to biotin, which allowed cells that bound the X probe to be further labeled post-hybridization with a streptavidin-fluorophor conjugate that emits in Channel 5 or Channel 6.

The hybridization efficiency of the FISH-IS procedure was evaluated by using spectrum green labeled probes for chromosome 8 and chromosome Y obtained from VYSIS (Dowers Groves, Ill.). The ImageStream data showed that 80% of the single sperm cells are chromosome 8 positive and about 50% of the single sperm cells contained chromosome Y.

### Example 4

### Human Jurkat Fish-IS

### Two Color FISH-IS: Probing Chromosome 8 and Y

Human Jurkat Cells: Preliminary experiments in the human system were performed using the Jurkat T cell line (ATCC, Manassas, Va.). This cell line is derived from a male and is expected to have two copies of chromosome 8 and one copy each of the X and Y chromosomes.

The Jurkat T cell line was cultured and maintained according to standard cell culture methods. The cells were harvested by low-speed centrifugation and then resuspended in phosphate-buffered saline (PBS) plus 1% fetal bovine serum (FBS). An equal volume of Caltag Fix and Perm Solution A was added to the resuspended cells for 15 min at room temperature. The cells were then washed with PBS-FBS and centrifuged at 740×g for 10 minutes. The cell pellets were washed with 100 μl PBS-FBS and centrifuged in a microfuge. The cells were resuspended in Hybridization Buffer (see Example 5) that contained the appropriate concentration of each probe as recommended by the manufacturer. The Chromosome Y probe that was used was labeled with Cy3, and the Chromosome 8 probe that was used was labeled with a fluorescein dye. The probes were hybridized with the cells by incubating the mixture at 65° C. for 2 h, then at 90° C. for 5 minutes, followed by 37° C. for at least 16 h. Following hybridization, the cells were washed for 20 min at 37° C., in 55% Formamide and 2×SSC.

When Jurkat cells were hybridized with the Chromosome Y-Cy3 probe, a strong signal was observed. Under the fluorescent microscope a strong signal was seen in nearly all cells with an exposure of 0.5 second.

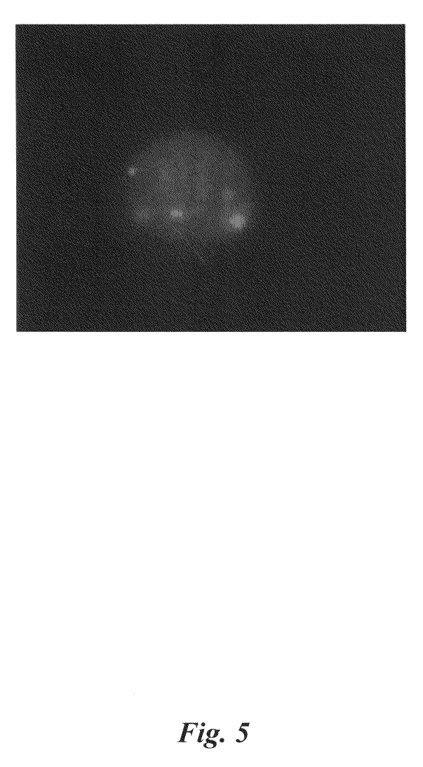

The fluorescein-labeled chromosome 8 probe dots in the Jurkat cells were visible in most cells. However, the intensity of the signal was not nearly as strong as with the Y chromosome probe. An exposure time of 1 second for the fluorescent microscope was often required. In order to increase the fluorescent label signal, a post-hybridization amplification of the signal was performed. The fluorescein signal was amplified by using an Alexa Fluor 488 labeled anti-fluorescein antibody followed by a secondary Alexa Fluor 488 labeled anti-isotype antibody. As shown in FIG. 5, this produced a stronger signal. Alexa Fluor 488 also emits in Channel 3 of the ImageStream and has a nearly identical spectrum to fluorescein; accordingly, both signals were observed in Channel 3, providing a stronger signal. Jurkat cells were analyzed using the probes for chromosome 8 and the Y chromosome. Microscopic examination showed that 80% of the observed cells had visible Cy3 dots while 50% had visible AF 488 dots.

Jurkat cells were hybridized with the biotin-labeled X chromosome probe. In single-probe trial experiments streptavidin-Alexa Fluor 488 was used to visualize the probe. In addition, a tyramide signal amplification strategy was investigated to amplify the Chromosome X signal. Horseradish peroxidase was used to generate many copies of a tyramide radical linked to another molecule. In this instance, the biotin-tyramide was used and detection was achieved with streptavidin attached to a fluorophore. A probe specific for Chromosome X was labeled with biotin and hybridized to Jurkat cells that were prepared as described above. To amplify the signal, following the hybridization reaction, a streptavidin-horseradish peroxidase conjugate was added to the biotin-labeled cells. Then the cells were exposed to tyramide conjugated to biotin, followed by incubation with Streptavidin-AF 488. AF-488 may be substituted with another appropriate fluorophore. This approach gave a more robust signal for X chromosome stained with Alexa Fluor 488.

Next, cells were probed for the X chromosome at the same time as probed for the Y chromosome and chromosome 8. Accordingly, a red or far red dye conjugated to streptavidin was used. An alternative approach to visualizing a 3^{rd} FISH-IS color for ImageStream channel #5 or 6 was investigated using Quantum Dot 705 (Quantum Dot Corporation, Hayward, Calif.) conjugated to streptavidin. The X chromosome was detected with this label as well by FITC.

### Analysis of Jurkat Cells

Jurkat cells were imaged using ImageStream Analysis platform. Approximately 20,000 cells were imaged. The cells were hybridized with a probe for Chromosome Y labeled with a fluorescent dye (Cy3) and Chromosome 8 labeled with fluorescent dye (Alexa Fluor 488). For the standard ImageStream configuration, having a standard fixed depth of field (11.5 micron), it was expected that only a small percentage of images would have in focus FISH spots because Jurkat cells have a diameter greater than 10 microns as well as nuclei greater than 5 microns.

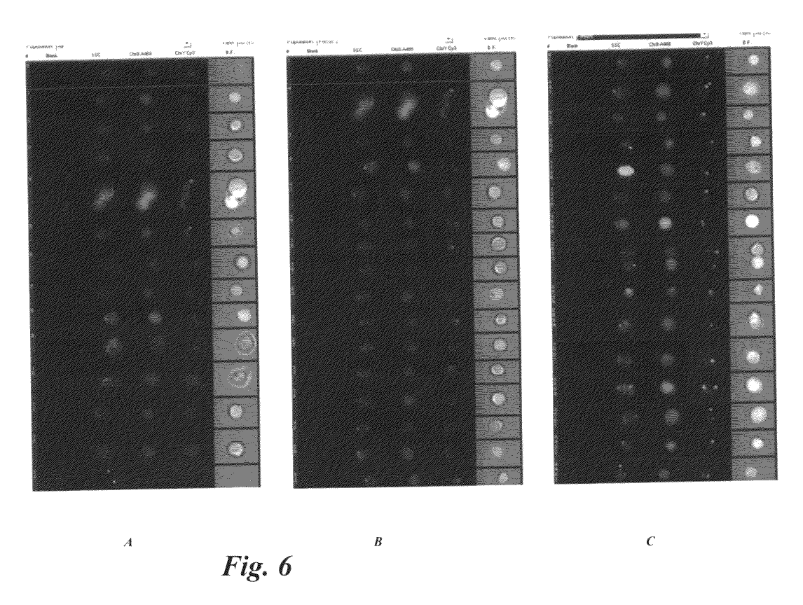

FIG. 6A depicts 11 separate cells shown top to bottom, each imaged sequentially in flow at a rate of 50 cells/second using four of the available 6 spectral channels of the standard ImageStream. From left to right the multimode channels correspond to (i) darkfield or side scatter channel (400-470 nm), (ii) Alexa 488 channel corresponding to the labeled 8 chromosome (500-560 nm), (iii) Cy3 channel corresponding to the labeled Y chromosome (560-595 nm), and (iv) brightfield (595-660 nm). Approximately 25% of the total number of the imaged cells (˜20,000 cells) were determined to be in focus. The focus determination was performed by plotting a feature (gradient max) derived from both darkfield and brightfield images in a scatterplot and gating on the in-focus population. FIG. 6 indicates that cell identification numbers 1, 4, 7, and 8 appeared to be the best in focus of the 11 cells displayed. After gating on only the cells in focus, FIG. 6B depicts 11 separate cells of the “in-focus” population. Similarly, a gate was determined for the best in-focus FISH spots as shown in FIG. 6C.

### Example 5

### Preparation of Cultured Cells for Fish-IS Analysis

An exemplary protocol for preparing somatic cells for FISH-IS is as follows. Cells are first washed with ice-cold 1×PBS (centrifuge at 1200 rpm for 5 min at 4° C.). After the final wash, the cells are centrifuged at 1400 rpm for 5 min at 4° C. Freshly-prepared ice-cold Carnoy's Fixative (3 part methanol: 1 part acetic acid; 0.4 ml/ml) is then added. The cells are removed from the fixative by centrifugation at 1400 rpm for 5 min at 4° C. The cells are resuspended in ice-cold 70% Carnoy's Fixative (0.4 ml/ml at 10⁷ cells/100 μl). (Cells can be stored at −20° C. for later use.) The cells are pelleted by centrifugation at 1200 rpm for 8 min at 4° C. and then resuspended in 30 μl of 2×SSC (pH 5.3). After centrifugation, the cells are resuspended in 7 μl of Vysis Hybridization Buffer (Vysis Inc., subsidiary of Abbott Laboratories, Downers Grove, Ill.), 2 μl water, 1 μl CEP probe or Chromosome Y (Cambio concentrated probe). After incubating the probe and the cells at 80° C. for 5 min, the cells are incubated at 42° C. for a period of time between 2 hr to overnight. Then 40 μl of 2×SSC (pH 7.0) is added to each sample. The cells are removed from the mixture by centrifugation. The cells are resuspended in 30 μl of 0.4×SSC/0.3% NP-40 and incubated at 73° C. for 2 min. Then 30 μl of 1×PBS with DAPI (10 μg) is added, followed by filtering the mixture through a 40 μm filter. The cells are then analyzed according to Image Stream 100 Analysis.

### Example 6

### Human Sperm Fish-IS

The labeling techniques developed using Jurkat cells in Example 4 were combined with the optimized non-aggregating sperm pre-hybridization techniques described in Example 1 and both applied to human sperm cells. The FISH-IS protocol described below was developed for human sperm.

The FISH-IS protocol for human sperm is as follows. Thaw human sperm samples and dispense the cells into 10 ml PBS-FCS. Centrifuge the cells at 720×g for 10 minutes, resuspend in DTT buffer at 10⁷ cells/ml (0.1 M hepes, pH 8.0; 50 mM NaCl, 0.1% Triton X-100, 1% FBS, 10 mM DTT), and incubate for 30 minutes at room temperature. Add an equal volume Caltag Fix and Perm Solution A for 15 minutes at room temperature. Centrifuge the cells at 720×g for 10 minutes. Sonicate the sperm cells for 10 minutes and then resuspend in PBS with 1% FBS, 1% Saponin, 0.1% Triton X-100 for 30 minutes room temperature. Centrifuge at 720×g for 10 minutes. Resuspend the cells in 100 μl PBS with 1% FBS, 1% Saponin. Centrifuge the cells in a microfuge at 400×g for 10 minutes and then resuspend in pre-denatured hybridization probe at 65° C. for 2 hours, 90° C. for 5 minutes, and at 37° C. for at least 16 hours. Add 10 volumes wash solution and incubate 10 minutes at 37° C.

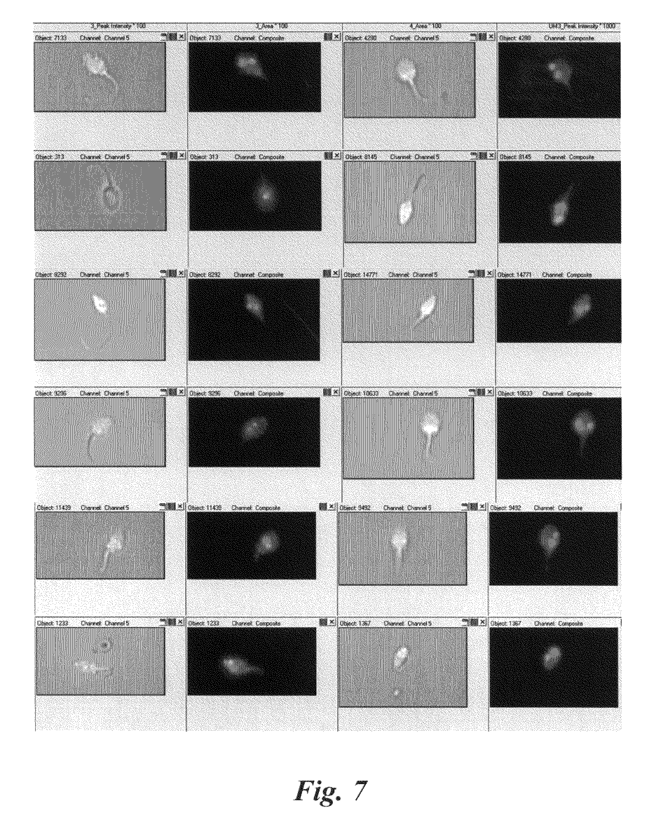

Sperm were first hybridized with the Cy3-labeled chromosome Y probe that gave the most robust signal in Jurkat cells (see Example 4). The percentage of sperm that display Y dots improved with the use of detergent during the dithiothreitol step. Post-hybridization amplification of the chromosome 8 FITC signal was performed as described in Example 4. All solutions used post-fixing included 1% Saponin unless otherwise stated. Small clumps of cells were dispersed by sonication for 10 minutes as needed. These FISHI-IS sperm cells were analyzed on the ImageStream as described herein (see FIGS. 7 and 11).

Human sperm cells were imaged using the ImageStream Analysis platform. The cells were hybridized with probes to Chromosomes Y labeled with Cy3 and Chromosomes 8 labeled with Alexa Fluor 488 consistent with the FISH-IS protocols outlined herein. The resulting ImageStream data file showed significant numbers of in-focus sperm with FISH spots. FIG. 7 illustrates an example of the sample imagery collected, twelve sperm images (brightfield and composite fluorescent spectra) that show single copies of the 8 and Y chromosomes.

### Example 7

### Procedure for Preparation of Human Sperm for Fish-IS Analysis

An exemplary method for fixing and permeabilizing human sperm followed by hybridization with a nucleic acid probe is provided as follows. Human sperm are thawed (0.4 ml) and ice-cold 6 mM EDTA in 1×PBS (final volume 6 ml) is added. The sperm cells are collected by centrifugation at 4° C., 10 min, 5000 rpm. The cells are resuspended in ice-cold 6 mM EDTA/PBS containing 10 mM DTT (1-3×10⁷ cells/ml) and incubated on ice for 30 min. The sperm are centrifuged at 4° C., 10 min, 5000 rpm and then resuspended in ice-cold 1×PBS (1-3×10⁷ cells/ml). Ice-cold Carnoy's (3:1; 0.4 ml/ml) is added dropwise while vortexing), keeping the sample on ice. The sperm cells are collected by centrifuge at 4° C., 10 min, 6000 rpm and then resuspended in ice-cold 60%, 70%, 80%, or 90% Carnoy's in PBS. The cells are pelleted by centrifugation at 4° C., 10 min, 6000 rpm and washed in 50 μl of 2×SSC (pH 5.3) and centrifuged as before. Hybridization is performed in Vysis Hybridization Buffer (see Example 5) with 100 μg probe. Hybridization proceeds at 80° C. for 5 min and then at 42° C. for 5-18 hr. After hybridization, 30 μl of 2×SSC (pH 7.0) is added and the sperm are centrifuged at 4° C., 10 min, 6000 rpm. The cells are resuspended in 30 μl of 0.4×SSC (pH 7.0), 0.3% NP-40 and then incubated at 73° C. for 2 min. To this mixture is added 20 μl 2×SSC (pH 7.0). The cells are then analyzed according to Image Stream 100 Analysis.

An alternative fixing procedure is as follows. For the second resuspension of the sperm cells in Carnoy's solution, the cells are resuspended in ice-cold 30% Carnoy's in PBS; centrifuged at 4° C., 10 min, 6000 rpm, washed in 50 μl of 2×SSC (pH 5.3), and again pelleted by centrifugation. The sperm cells are resuspended in 1% paraformaldehyde on ice for 5 min. 2×SSC (pH 7.0) is added and the cells are collected by centrifugation at 4° C., 10 min, 6000 rpm. Hybridization proceeds as described above.

### Example 8

### Murine Sperm Fish-IS

Mouse sperm were obtained by removing epididymi from male mice and sperm were allowed to swim out after longitudinal cuts. Epididymi were removed from BALB/c or CD-1 mice, and sperm were collected during two 30 minute incubations at 32° C. in FBS or 2.2% sodium citrate. Sperm were first treated with trypsin to remove long tails. After DNA decondensation, sperm were fixed in various concentrations of Camoy's solution and hybridized using the same procedures as for human sperm. It was found that other cells contaminated the murine sperm preparations but these were easily distinguished due to differences in size and morphology using the ImageStream IDEAS Analysis platform. The murine sperm tended to be longer and narrower than human sperm prepared with the same procedures. In some experiments, the yield of single sperm cells was as high as 95% of the original cell concentration.

Centromeric point probes are not available commercially for the mouse chromosomes. Chromosome-specific probes were obtained for chromosomes 8, X, and Y as plasmids from Dr. Andrew Wyrobek's Laboratory at Lawrence Livermore National Laboratory (Livermore, Calif.). These probes are described in, for example, Boyle and Ward, Genomics 12:517-525 (1992) and Disteche et al., Cytogenet. Cell Genet. 39:262 (1985). Plasmids were transformed into E. coli and harvested using Qiagen Qiaprep Miniprep Kit. Probes were labeled using Universal Linkage System (ULS) reagents developed by Kreatech (Amsterdam, Netherlands) according to the manufacturer's protocol. The ULS Alexa Fluor 488 reagent was obtained from Molecular Probes (Eugene, Oreg.), and the ULS-Cy3 reagent was obtained from Amersham (Piscataway, N.J.). DNA was digested with DNase I, labeled, and purified according to manufacturer's instructions. One μg of DNA was used per labeling reaction and 100 ng was used per hybridization. A Chromosome Y “paint” probe labeled with Cy 3 was obtained from Cambio. Hybridization buffer contained 55% formamide, 1×SSC, and 1% saponin.

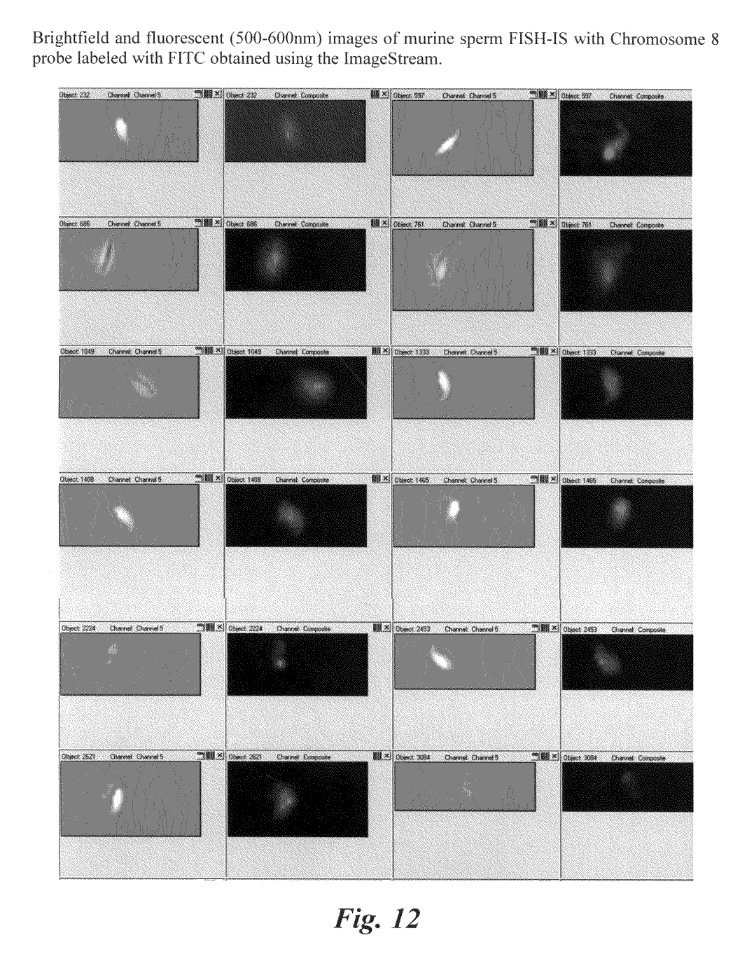

Probes were tested on the male mouse macrophage cell line, RAW. A strong signal was seen with the Cy 3-labeled chromosome Y “paint” probe. When the X chromosome probe was labeled with Cy 3, nearly all cells observed under the microscope were labeled but they were not as bright as cells labeled with the Chromosome Y probe. A chromosome 8 probe was labeled with Alexa Fluor 488 using the ULS system. After hybridization, sperm were analyzed on the ImageStream, and the results are presented in FIG. 12. Distinct AF488 FISH spots were seen in Channel 3 (500-600 nm).

### Example 9

### Preparation of Murine Sperm for Fish-IS

An exemplary method for fixing and permeabilizing mouse sperm followed by hybridization with a nucleic acid probe is provided as follows. Mouse sperm are collected in 2.2% Na Citrate followed by centrifugation at 4° C., for 10 min at 7000 rpm. Sperm cells are resuspended in 5 ml 1×PBS with trypsin (100-500 μg/ml) at room temperature for 2-5 min. The cells are then homogenized in a Dounce tissue grinder (˜4 times). To the cells 1 ml FCS is added and the cells are then centrifuged at 4° C., 10 min, 7000 rpm, followed by a wash with 1×PBS. The sperm cells are resuspended in ice-cold 6 mM EDTA/PBS with 10 mM DTT (1-3×10⁷ cells/ml). After an incubation on ice for 30 min, the cells are centrifuged at 4° C., 10 min, 7000 rpm. The pelleted sperm cells are resuspended in ice-cold 1×PBS (1-3×10⁷ cells/ml). Ice-cold Carnoy's (3:1; 0.4 ml/ml) is added dropwise while vortexing. The cells are maintained on ice for 30 min and then centrifuged at 4° C., 10 min, 7000 rpm, and then resuspended in ice-cold 30-70% Carnoy's in PBS, which is added dropwise while vortexing. The sperm cells are then stored at −20° C. After thawing, the fixed mouse sperm (1˜5×10⁶ cells/ml) are centrifuged at 4° C., 10 min, 3000 rpm. After awash in 50 μl of 2×SSC (pH 5.3), the sperm are pelleted by centrifugation at 4° C., 10 min, 3000 rpm. Hybridization is performed in Hybridization Buffer (50% formamide, 10% Dextran sulfate, 2×SSC, 1 μg Cot-1 DNA) with 100 μg probe. Hybridization proceeds at 80° C. for 5 min and then at 42° C. overnight.

All of the above U.S. patents, U.S. patent application publications, U.S. patent applications, foreign patents, foreign patent applications, and non-patent publications referred to in this specification and/or listed in the Application Data Sheet, are incorporated herein by reference, in their entirety.

Those skilled in the art will recognize, or be able to ascertain, using no more than routine experimentation, many equivalents to the specific embodiments of the invention described herein. Such equivalents are intended to be encompassed by the following claims.

1. A method for identifying a sperm cell in a biological sample, comprising: (a) directing brightfield and laser light at a cell; (b) obtaining a side scatter profile and brightfield image using a CCD detector; and (c) determining the spatial content of the side scatter profile and brightfield image, and therefrom identifying a sperm cell.

2. The method of claim 1 wherein relative movement exists between the cell and the detector.

3. The method according to claim 1 wherein the biological sample comprises a heterogeneous cell population.

4. The method according to claim 1 further comprising multispectral imaging.

5. The method according to claim 4 wherein multispectral imaging comprises (a) detecting in a first imaging channel a first nucleic acid probe that is hybridized to a first target chromosomal DNA sequence, wherein the first probe is attached to a first fluorochrome; and (b) detecting in a second imaging channel a second nucleic acid probe that is hybridized to a second target chromosomal DNA sequence, wherein the second probe is attached to a second fluorochrome.

6. The method according to claim 5 further comprising (a) determining a system mask area to user mask area ratio (first ratio) of the first fluorochrome detected in the first imaging channel; and (b) determining a system mask area to user mask area ratio of the second fluorochrome detected in the second imaging channel (second ratio).

7. The method according to claim 6 further comprising plotting the first ratio against the second ratio on a bivariate scatter plot.

8. The method according to claim 4 wherein multispectral imaging comprises (a) detecting in a first imaging channel a first nucleic acid probe that is hybridized to a first target chromosomal DNA sequence, wherein the first probe is attached to a first fluorochrome; and (b) detecting in a second imaging channel a second nucleic acid probe that is hybridized to a second target chromosomal DNA sequence, wherein the second probe is attached to a second fluorochrome; wherein multispectral imaging comprises (c) detecting in a third imaging channel a third nucleic acid probe that is hybridized to a third target chromosomal DNA sequence, wherein the third probe is attached to a first fluorochrome; and (b) detecting in a fourth imaging channel a fourth nucleic acid probe that is hybridized to a fourth target chromosomal DNA sequence, wherein the fourth probe is attached to a second fluorochrome.

9. The method according to claim 8 further comprising (a) determining a system mask area to user mask area ratio (first ratio) of the first fluorochrome detected in the first imaging channel; (b) determining a system mask area to user mask area ratio of the second fluorochrome detected in the second imaging channel (second ratio); (c) determining a system mask area to user mask area ratio (third ratio) of the third fluorochrome detected in the third imaging channel; (d) determining a system mask area to user mask area ratio of the fourth fluorochrome detected in the fourth imaging channel (fourth ratio).
